(19) United States
(12) Patent Application Publication
(10) Pub. No.: US 2024/0032987 A1
(43) Pub. Date: Feb. 1, 2024

(54) SURGICAL INSTRUMENTS, SYSTEMS, AND METHODS INCORPORATING ULTRASONIC AND ELECTROSURGICAL FUNCTIONALITY

(71) Applicant: Covidien LP, Mansfield, MA (US)

(72) Inventors: Michael B. Lyons, Boulder, CO (US); James E. Dunning, Boulder, CO (US); Kristen P. Bradley, Westminster, CO (US); David J. Van Tol, Boulder, CO (US); Christopher T. Tschudy, Arvada, CO (US); Keith W. Malang, Longmont, CO (US); Donald L. Tonn, Superior, CO (US); Mark A. Johnston, Boulder, CO (US); James R. Fagan, Erie, CO (US)

(21) Appl. No.: 18/266,148

(22) PCT Filed: Dec. 2, 2021
(86) PCT No.: PCT/US2021/061631

§ 371 Date: Jun. 8, 2023

Related U.S. Application Data
(60) Provisional application No. 63/122,633, filed on Dec. 8, 2020.

(51) Int. Cl. A61B 18/14; A61B 17/32

(52) U.S. Cl. CPC A61B 18/1445; A61B 17/320092; A61B 2017/00026

(57) ABSTRACT

A method of treating tissue includes clamping tissue between an ultrasonic blade and a jaw member and simultaneously: transmitting ultrasonic energy to the ultrasonic blade to vibrate the ultrasonic blade at a first blade velocity thereby heating the clamped tissue; and supplying electrosurgical energy, at a constant voltage, to the jaw member and the ultrasonic blade at different potentials such that the electrosurgical energy is conducted therebetween and through the clamped tissue to heat the clamped tissue. An impedance of the clamped tissue is monitored and the simultaneous transmission of ultrasonic energy and supply of electrosurgical energy is terminated and/or a notification is output once the clamped tissue is sealed, as indicated by the impedance of the clamped tissue being equal to or greater than a threshold impedance. Surgical instruments and end effectors thereof for treating tissue in this manner are also provided.

### CROSS-REFERENCE TO RELATED APPLICATIONS

This application is a 371 National Stage Application of International Application No. PCT/US2021/061631, filed Dec. 2, 2021, which claims the benefit of, and priority to, U.S. Provisional Patent Application No. 63/122,633 filed on Dec. 8, 2020, the entire contents of which are hereby incorporated herein by reference.

### FIELD

The present disclosure relates to energy-based surgical instruments and, more particularly, to surgical instruments, systems, and methods incorporating ultrasonic and electrosurgical functionality to facilitate treating tissue, e.g., sealing and/or transecting tissue, and/or tissue sensing.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to coagulate, cauterize, fuse, seal, cut, and/or desiccate tissue to effect hemostasis. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical devices transmit Radio Frequency (RF) energy through tissue to treat tissue. An electrosurgical device may include, for example, opposing structures operable to clamp tissue therebetween and conduct energy, e.g., bipolar RF energy, through clamped tissue to treat, e.g., seal, the clamped tissue, or may include a monopolar probe configured to supply energy, e.g., monopolar RF energy, to tissue to treat, e.g., transect, tissue, while the energy is returned by a remote return electrode device. Additional or alternative electrosurgical devices, in either a monopolar or bipolar RF configuration, conduct RF through tissue to sense one or more properties.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a method of treating tissue including clamping tissue between an ultrasonic blade and a jaw member, simultaneously transmitting ultrasonic energy and supplying electrosurgical energy, monitoring an impedance of the clamped tissue during the simultaneous transmission of ultrasonic energy and supply of electrosurgical energy, and terminating the simultaneous transmission of ultrasonic energy and supply of electrosurgical energy when the clamped tissue is sealed. The ultrasonic energy is transmitted to the ultrasonic blade to vibrate the ultrasonic blade at a first blade velocity, thereby heating the clamped tissue. The electrosurgical energy is supplied, at a constant voltage, to the jaw member and the ultrasonic blade at different potentials such that the electrosurgical energy is conducted therebetween and through the clamped tissue to heat the clamped tissue. Completion of sealing of the clamped tissue is indicated by the impedance of the clamped tissue being equal to or greater than a threshold impedance.

In an aspect of the present disclosure, the first blade velocity is from about 2.4 m/s to about 5.0 m/s, from about 3.0 m/s to about 4.2 m/s, or about 3.6 m/s.

In another aspect of the present disclosure, the constant voltage is an applied voltage of from about 20 Vrms to about 45 Vrms; in other aspects, from about 25 Vrms to about 40 Vrms; and in still other aspects, from about 30 Vrms to about 35 Vrms.

In still another aspect of the present disclosure, the method further includes, after terminating the simultaneous transmission of ultrasonic energy and supply of electrosurgical energy, transmitting ultrasonic energy to the ultrasonic blade to vibrate the ultrasonic blade at a second blade velocity greater than the first blade velocity to transect the sealed tissue.

In yet another aspect of the present disclosure, the second blade velocity is from about 7.0 m/s to about 10.0 m/s, from about 7.5 m/s to about 8.5 m/s, or about 8.0 m/s.

In still yet another aspect of the present disclosure, the method further includes terminating the transmission of ultrasonic energy to vibrate the ultrasonic blade at the second blade velocity when it is determined that transection of the sealed tissue is complete.

In another aspect of the present disclosure, the jaw member includes a body defining first and second radiused surfaces and a jaw liner defining a tissue contacting surface disposed between the first and second radiused surfaces. The tissue contacting surface opposes the ultrasonic blade when clamping tissue therebetween. In such aspects, supplying the electrosurgical energy includes conducting the electrosurgical energy between the ultrasonic blade and the first and second radiused surfaces.

In another aspect of the present disclosure, the first and second radiused surfaces define radii of curvature of from about 0.003 inches to about 0.012 inches, from about 0.005 inches to about 0.010 inches, or about 0.008 inches.

In yet another aspect of the present disclosure, a first plane is tangential to the first and second radiused surfaces and the tissue contacting surface defines a second plane. The second plane is recessed relative to the first plane a distance of from about 0.001 inches to about 0.010 inches, from about 0.002 inches to about 0.005 inches, or about 0.003 inches.

In still another aspect of the present disclosure, the ultrasonic blade defines a tissue contacting surface having first and second angled or arcuate surface portions meeting at an apex configured to oppose the jaw member when clamping tissue therebetween.

Also provided in accordance with aspects of the present disclosure is an end effector assembly of a surgical instrument. The end effector assembly includes an ultrasonic blade adapted to receive ultrasonic energy to vibrate the ultrasonic blade at a blade velocity and adapted to connect to a source of electrosurgical energy at a first potential. The end effector assembly further includes a jaw member movable relative to the ultrasonic blade from a spaced-apart position to an approximated position for clamping tissue therebetween. The jaw member includes a structural body including first and second uprights extending longitudinally along the structural body in spaced-apart relation relative to one another. The first and second uprights define radiused free ends and are adapted to connect to the source of electrosurgical energy at a second potential. A first plane is defined tangential to the radiused free ends of the first and second uprights. The jaw member further includes a jaw liner disposed within the structural body between the first and second uprights. The jaw liner defines a tissue contacting surface positioned to oppose the ultrasonic blade in the approximated position. The tissue contacting surface defines a second plane recessed relative to the first plane.

The ultrasonic blade and/or the jaw member may be configured similar to any of the aspects detailed hereinabove or otherwise herein.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1A illustrates a surgical system provided in accordance with the present disclosure including a surgical instrument, a surgical generator and, in some aspects, a return electrode device;

FIG. 1B is a perspective view of another surgical system provided in accordance with the present disclosure including a surgical instrument;

FIG. 1C is a schematic illustration of a robotic surgical system provided in accordance with the present disclosure;

FIG. 2A is an enlarged, side, perspective view of a distal portion of an end effector assembly configured for use with the surgical instrument of FIG. 1A, the surgical instrument of FIG. 1B, the robotic surgical system of FIG. 1C, or any other suitable surgical instrument or system;

FIG. 2B is a transverse, cross-sectional view of the end effector assembly of FIG. 2A;

FIG. 3A is an enlarged, side, perspective view of the jaw member of the end effector assembly of FIG. 2A;

FIG. 3B is an enlarged, side, perspective view of another jaw member configured for use with the end effector assembly of FIG. 2A;

FIG. 3C is a transverse, cross-sectional view of still another jaw member configured for use with the end effector assembly of FIG. 2A;

FIG. 4 is a transverse, cross-sectional view of the elongated assembly of the surgical instrument of FIG. 1A;

FIGS. 5A and 5B are graphs representing energy delivery signals as a function of time in accordance with aspects of the present disclosure;

FIG. 6 is a flow diagram illustrating a method of sealing tissue in accordance with the present disclosure;

FIGS. 7A and 7B are flow diagrams illustrating methods of transecting tissue subsequent to tissue sealing in accordance with the present disclosure;

FIG. 8 is a plot of experimental results of burst pressure of a sealed vessel as a function of the vessel size, the ultrasonic energy setting (e.g., waveguide velocity), and the electrosurgical energy setting; and

FIG. 9 is a plot of experimental results of activation time required to seal a vessel as a function of the vessel size, the ultrasonic energy setting (e.g., waveguide velocity), and the electrosurgical energy setting.

### DETAILED DESCRIPTION

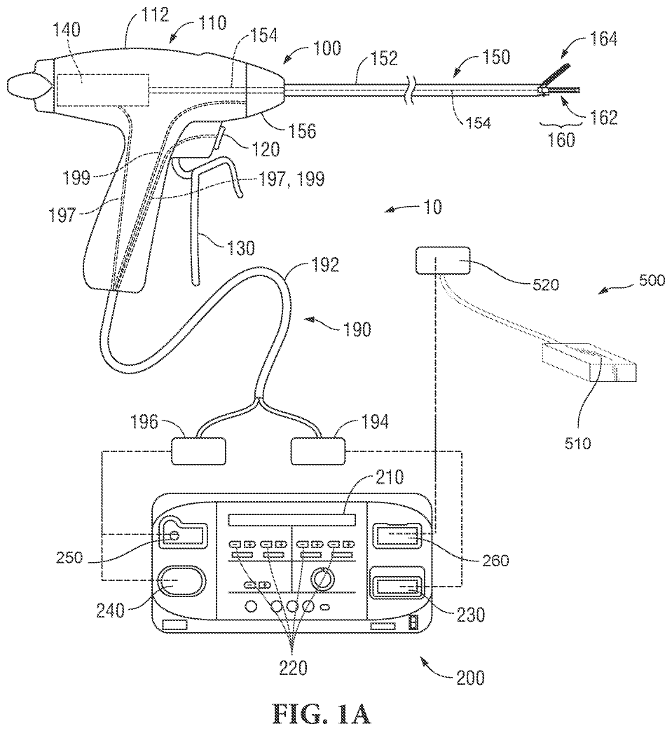

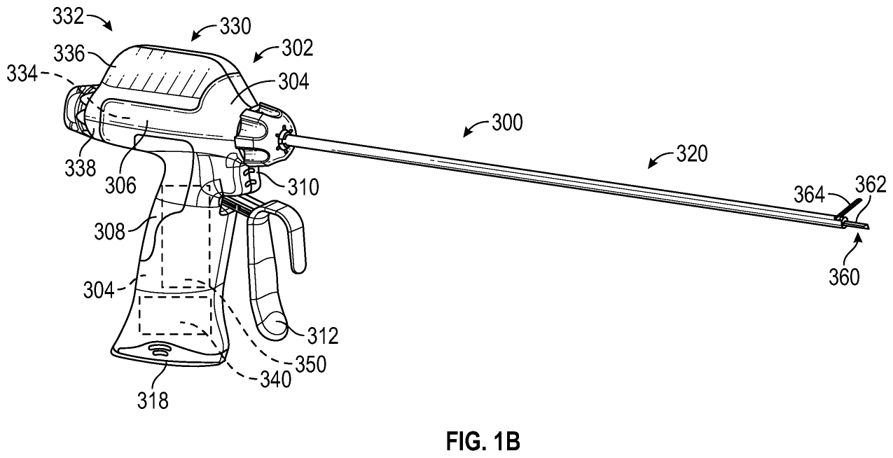

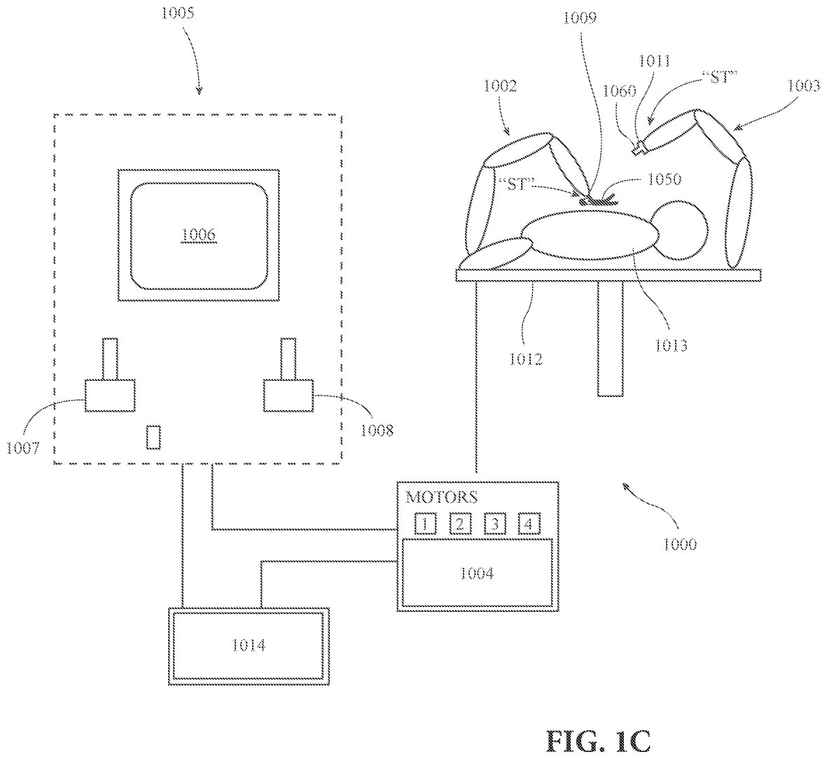

Referring to FIG. 1A, a surgical system provided in accordance with aspects of the present disclosure is shown generally identified by reference numeral 10 including a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system such as, for example, robotic surgical system 1000 (FIG. 1C) detailed below.

Surgical generator 200 includes a display 210, a plurality user interface features 220, e.g., buttons, touch-screens, switches, etc., an ultrasonic plug port 230, a bipolar electrosurgical plug port 240 and, in some aspects, active and return monopolar electrosurgical plug ports 250, 260, respectively. Surgical generator 200 is configured to produce ultrasonic drive signals for output through ultrasonic plug port 230 to surgical instrument 100 to activate surgical instrument 100 in an ultrasonic mode of operation and to provide electrosurgical energy, e.g., RF bipolar energy, for output through bipolar electrosurgical plug port 240 and/or RF monopolar energy for output through active monopolar electrosurgical port 250 to surgical instrument 100 to activate surgical instrument 100 in an electrosurgical mode of operation. It is also contemplated that one or more common ports (not shown) may be configured to act as any two or more of ports 230-260. In monopolar configurations, plug 520 of return electrode device 500 is connected to return monopolar electrosurgical plug port 260.

Continuing with reference to FIG. 1A, handle assembly 110 includes a housing 112 defining a body portion and a fixed handle portion. Handle assembly 110 further includes an activation button 120 and a clamp trigger 130. The body portion of housing 112 is configured to support an ultrasonic transducer 140. Ultrasonic transducer 140 may be permanently engaged with the body portion of housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different modes, e.g., a first actuated position corresponding to a first mode and a second actuated position corresponding to a second mode. In still other configurations, separate activation buttons may be provided, e.g., a first actuation button for activating a first mode and a second activation button for activating a second mode.

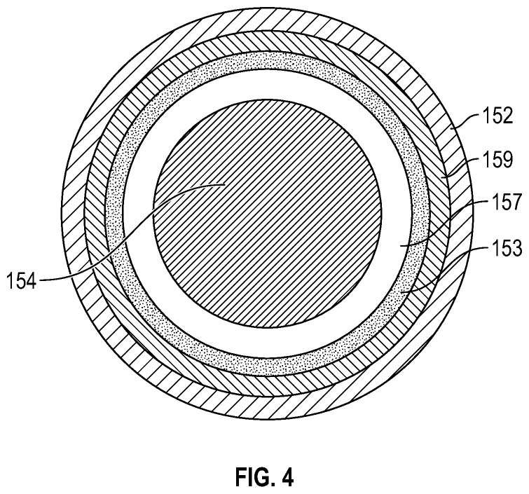

Elongated assembly 150 of surgical instrument 100 includes an outer drive sleeve 152, an inner support sleeve 153 (FIG. 4) disposed within outer drive sleeve 152, a waveguide 154 extending through inner support sleeve 153 (FIG. 4), a drive assembly (not shown), a rotation knob 156, and an end effector assembly 160 including a blade 162 and a jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110, a distal portion of outer drive sleeve 152 is operably coupled to jaw member 164, and a distal end of inner support sleeve 153 (FIG. 4) pivotably supports jaw member 164. As such, clamp trigger 130 is selectively actuatable to thereby move outer drive sleeve 152 about inner support sleeve 153 (FIG. 4) to pivot jaw member 164 relative to blade 162 of end effector assembly 160 from a spaced-apart position to an approximated position for clamping tissue between jaw member 164 and blade 162. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

The drive assembly may be tuned to provide a specific jaw clamping force, or jaw clamping force within a jaw clamping force range, to tissue clamped between jaw member 164 and blade 162 or may include a force-limiting feature whereby the clamping force applied to tissue clamped between jaw member 164 and blade 162 is limited to a particular jaw clamping force or a jaw clamping force within a jaw clamping force range. The jaw clamping force, measured at a distance of about 0.192 inches from a distal end of jaw member 164 when clamp trigger 130 is fully actuated, may be from about 2 lbf to about 7 lbf, in other aspects from about 2.5 lbf to about 6.0 lbf, and, in still other aspects, about 3.2 lbf. Alternatively, the jaw clamping force may be about 5.5 lbf.

Waveguide 154, as noted above, extends from handle assembly 110 through the inner support sleeve. Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s). Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode of operation. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 such that, as detailed below, bipolar electrosurgical energy may be conducted between blade 162 and jaw member 164. In monopolar configurations, an electrical lead wire 199 is electrically coupled to waveguide 154 such that, as also detailed below, monopolar electrosurgical energy may be supplied to tissue from blade 162.

Alternatively, an electrical lead wire 199 may electrically couple to jaw member 164 in the monopolar configuration to enable monopolar electrosurgical energy to be supplied to tissue from jaw member 164. One or more second electrical lead wires 199 is electrically coupled to activation button 120 to enable the selective supply of electrosurgical energy from surgical generator 200 to waveguide 154 and/or jaw member 164 upon activation of activation button 120 in an electrosurgical mode of operation.

Referring to FIG. 1B, another surgical system provided in accordance with the present disclosure includes a surgical instrument 300. Surgical instrument 300 is similar to and may include any of the features of surgical system 10 (FIG. 1A) except that, rather than providing a separate surgical instrument and surgical generator tethered to one another via a cable assembly, surgical instrument 300 is cordless in that it incorporates an ultrasonic transducer and generator assembly (“TAG”) 330 as well as an electrosurgical generator 340 and a power source, e.g., a battery assembly 350, thereon or therein. In this manner, surgical instrument 300 is not required to be connected to a separate generator(s) or power source. Surgical instrument 300 is configured for use in an ultrasonic mode of operation and an electrosurgical mode of operation.

As surgical instrument 300 is similar to and may include any of the features of surgical system 10 (FIG. 1A), only differences therebetween are described in detail below while similarities are summarily described or omitted entirely. Surgical instrument 300 includes a handle assembly 302 and an elongated assembly 320 extending distally from handle assembly 302. Handle assembly 302 includes a housing 304 defining a body portion 306 and a fixed handle portion 308. Handle assembly 302 further includes an activation button 310 and a clamp trigger 312. Elongated assembly 320 has an end effector assembly 360 at a distal end portion thereof including an ultrasonic blade 362 and a jaw member 364.

Body portion 306 of housing 304 is configured to support TAG 330 thereon or therein. TAG 330 includes an ultrasonic generator 332 and an ultrasonic transducer 334. TAG 330 may be permanently engaged with body portion 306 of housing 304 or removable therefrom. Ultrasonic generator 332 includes a housing 336 configured to house the internal electronics of ultrasonic generator 332, and a cradle 338 configured to rotatably support ultrasonic transducer 334.

Fixed handle portion 308 of housing 304 defines a compartment 314 configured to receive electrosurgical generator 340 and battery assembly 350 and a door 318 configured to enclose compartment 314. Electrosurgical generator 340 and battery assembly 350 may be integrally formed, releasably engaged, or separate from one another and, are configured for releasably receipt within compartment 314, accessible via door 318. As an alternative to electrosurgical generator 340 being insertable into compartment 314, electrosurgical generator 340 may be mounted, e.g., permanently or releasably, to an exterior of fixed handle portion 308, e.g., depending therefrom, or may be disposed on or within housing 336 of TAG 330 (permanently or removably).

Electrical connections (not shown) within housing 304 of handle assembly 302 serve to electrically couple activation button 310 and/or battery assembly 350 when surgical instrument 300 is assembled for use. In some configurations, surgical instrument 300 may be utilized without electrosurgical generator 340, thus functioning only in the ultrasonic mode of operation. Additionally or alternatively, surgical instrument 300 may be utilized without TAG 330, thus functioning only in the electrosurgical mode of operation. Surgical instrument 100 (FIG. 1A) may likewise operate in this manner, where ultrasonic plug 194 or electrosurgical plug 196 are not connected to generator 200 (see FIG. 1A).

With reference to FIG. 1C, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be ultrasonic surgical instrument 100 (FIG. 1A), e.g., configured for use in both an ultrasonic mode of operation and an electrosurgical (bipolar or monopolar) mod of operation, wherein manual actuation features, e.g., actuation button 120 (FIG. 1A), clamp lever 130 (FIG. 1A), etc., are replaced with robotic inputs. In such configurations, robotic surgical system 1000 may include or be configured to connect to an ultrasonic generator, an electrosurgical generator, and a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

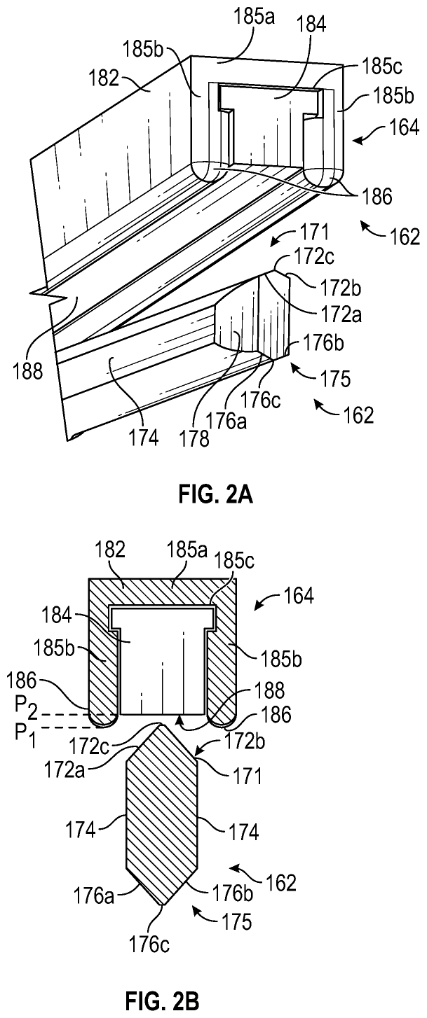

Referring to FIGS. 2A and 2B, end effector assembly 160 of surgical instrument 100 of surgical system 10 (FIG. 1A) is detailed; although end effector assembly 160 may be utilized with any other suitable surgical instrument and/or surgical system. Further, end effector assembly 360 of surgical instrument 300 (FIG. 1B) may include any or all of the features of end effector assembly 160.

End effector assembly 160 includes a blade 162 and a jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, although one configuration of blade 162 is described and illustrated herein, it is contemplated that blade 162 may additionally or alternatively be formed to include any suitable features, e.g., a tapered configuration, various different cross-sectional configurations along its length, cut-outs, indents, edges, protrusions, straight surfaces, curved surfaces, angled surfaces, wide edges, narrow edges, and/or other features.

In embodiments, blade 162 defines a generally convex first tissue contacting surface 171, e.g., the surface that opposes jaw member 164 in the approximated position thereof. Generally convex first tissue contacting surface 171 may be defined by a pair of surfaces 172a, 172b (flat or arcuate, e.g., convex, surfaces) that converge at an apex 172c, or may be formed by a continuously arcuate surface defining an apex 172c. Blade 162 may further define substantially flat lateral surfaces 174 (excluding any curvature due to the curvature of blade 162 itself) on either side of first tissue contacting surface 171, and a second tissue contacting surface 175 opposite first tissue contacting surface 171 and similarly configured relative thereto, e.g., with surfaces 176a, 176b (or surface portions) converging at an apex 176c, although other configurations are also contemplated.

Waveguide 154 (FIG. 1A), or at least the portion of waveguide 154 proximally adjacent blade 162, may define a cylindrical-shaped configuration. Plural tapered surfaces (not shown) may interconnect the cylindrically-shaped waveguide 154 with the polygonal (or rounded-edge polygonal) configuration of blade 162 to define smooth transitions between the body of waveguide 154 and blade 162. Additionally or alternatively, inwardly tapered surfaces 178 may extend from lateral surfaces 174 at the distal end of blade 162 such that the distal end of blade 162 defines a narrowed configuration as compared to the body of blade 162.

First tissue contacting surface 171 is configured to contact tissue clamped between blade 162 and jaw member 164 for treating clamped tissue, e.g., sealing and/or transecting clamped tissue, while second tissue contacting surface 175 may be utilized for, e.g., tissue transection, back scoring, etc. The distal end of blade 162 and/or some or all of the other surfaces of blade 162 may additionally or alternatively be utilized to treat tissue.

Lateral surfaces 174 and, in aspects, tapered surfaces 178 and/or the proximal tapered surfaces (not shown), may be coated with an electrically insulative material such that, in the electrosurgical mode of operation, current is directed from first tissue contacting surface 171 of blade 162 to jaw member 164 rather than from lateral surfaces 174 (or tapered surfaces 178 or the proximal tapered surfaces (not shown)). Suitable electrically insulative coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

Blade 162 may define a maximum width between lateral surfaces 174, at the proximal end portions thereof, of from about 0.60 inches to about 0.70 inches; and in other aspects, of about 0.65 inches. Blade 162 and may taper in width in a proximal-to-distal direction along at least a portion of a length thereof to a minimum width between lateral surfaces 174 and/or at the distal end of blade 162, of from about 0.27 inches to about 0.33 inches; and in other aspects, of about 0.30 inches. The apexes 172c, 176c of blade 162 may define surfaces having widths of from about 0.000 inches to about 0.010 inches; and, in other aspects, of about 0.003 inches.

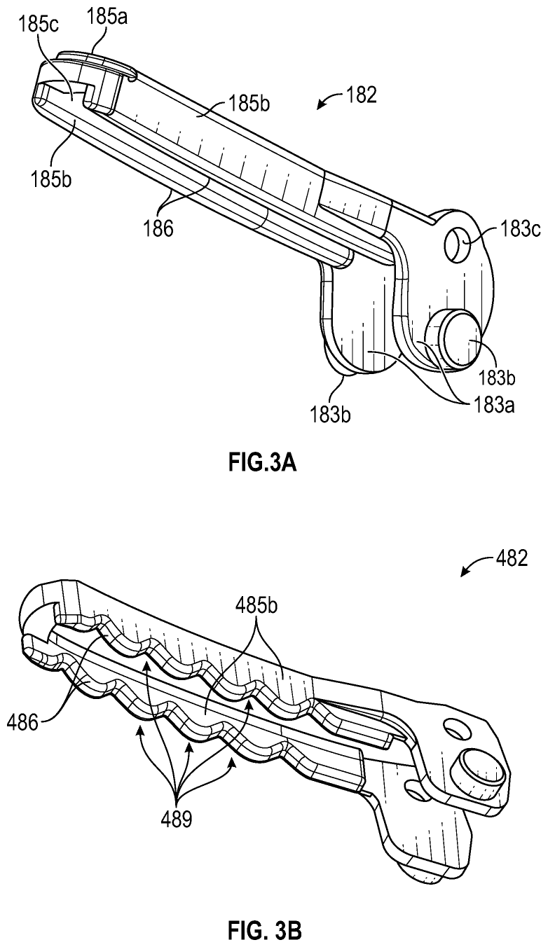

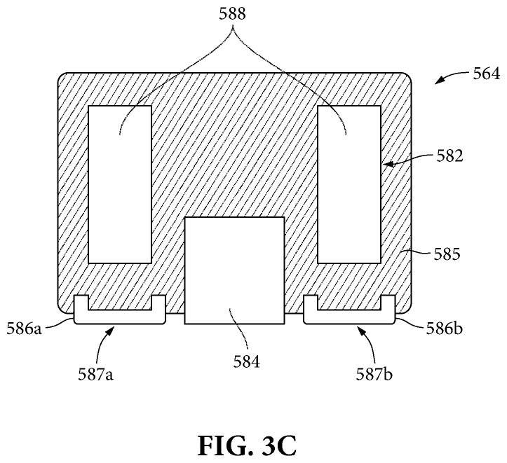

With additional reference to FIG. 3A, jaw member 164 of end effector assembly 160 includes a more-rigid structural body 182 and a more-compliant jaw liner 184. Structural body 182 may be formed from an electrically conductive material, e.g., stainless steel, or may include electrically conductive portions. Structural body 182 includes a pair of proximal flanges 183a that are pivotably coupled to the inner support sleeve 153 (FIG. 4) of surgical instrument 100 (FIG. 1A) via receipt of pivot bosses 183b of proximal flanges 183a within corresponding openings (not explicitly shown) defined within the inner support sleeve 153 (FIG. 4) and operably coupled with outer drive sleeve 152 (FIGS. 1A and 4) via a drive pin (not shown) secured relative to outer drive sleeve 152 and pivotably received within apertures 183c defined within proximal flanges 183a. As such, sliding of outer drive sleeve 152 (FIGS. 1A and 4) about inner support sleeve 152 (FIG. 4) pivots jaw member 164 relative to blade 162 from a spaced-apart position to an approximated position to clamp tissue between jaw liner 184 of jaw member 164 and blade 162.

Structural body 182 of jaw member 164 further includes an elongated distal portion defining a generally U-shaped configuration including a backspan 185a and a pair of spaced-apart uprights 185b extending from backspan 185a in generally perpendicular orientation relative to backspan 185a and generally parallel orientation relative to one another. Backspan 185a and uprights 185b cooperate to define a cavity 185c therein. Cavity 185c defines an elongated, generally T-shaped configuration for slidable receipt and retention of jaw liner 184 therein, although other suitable configurations for receiving and retaining jaw liner 184 are also contemplated.

Structural body 182 is adapted to connect to a source of electrosurgical energy and, in a bipolar electrosurgical mode of operation, is charged to a different potential as compared to blade 162 to enable the conduction of bipolar electrosurgical (e.g., RF) energy therebetween, through tissue clamped therebetween, to treat the tissue. More specifically, bipolar electrosurgical energy is configured to flow between first tissue contacting surface 171 of blade 162 and free ends 186 of uprights 185b of structural body 182 and through tissue disposed therebetween to complete the electrosurgical energy circuit. In a monopolar electrosurgical mode of operation, structural body 182 may be un-energized, may be charged to the same potential as compared to blade 162, or may be energized while blade 162 is not energized.

Free ends 186 of uprights 185b of structural body 182 define radiused edges to inhibit current concentrations and facilitate the conduction of energy between free ends 186 and blade 162. More specifically, free ends 186 may define radii of curvature of from about 0.003 inches to about 0.012 inches; in other aspects, from about 0.005 inches to about 0.010 inches; and, in still other aspects, of about 0.008 inches. Other suitable raidused or other configurations are also contemplated, as are other surface features such as, for example, teeth, scallops, etc. to facilitate tissue grasping and retention (see, e.g., FIG. 3B).

Referring to FIG. 3B, in other configurations, the jaw member may include a more-rigid structural body 482 similar to and including any of the features of structural body 182 (FIG. 3A) except that the free ends 486 of uprights 485b of structural body 482 define scallops 489 extending longitudinally along at least portions of the lengths of uprights 485b. Teeth or other suitable tissue-gripping and retention features are also contemplated. The extent to which free ends 486 of uprights 485b extend relative to the jaw liner may be measured from the apexes of scallops 489, the nadirs thereof, the midpoints thereof, or in any other suitable manner.

Turning to FIG. 3C, the structural body may alternatively embedded in an insulative material, e.g., an overmolded plastic. In such embodiments, electrically-conductive plates may be disposed on or captured by the overmolded plastic to function as the free ends of the uprights and enable electrical conduction of energy. More specifically, FIG. 3C illustrates another jaw 564 configured for use with end effector assembly 160 (FIGS. 2A and 2B) or other suitable end effector assembly includes a more-rigid structural body 582, a more-compliant jaw liner 584, an insulative housing 585, and first and second electrically-conductive plates 586a, 586b defining respective tissue-contacting surfaces 587a, 587b.

Structural body 582 includes a pair of proximal flanges (not shown) and an elongated distal portion defining a pair of spaced-apart upright supports 588 which may be separate from one another along the lengths thereof or joined via a backspan (not shown) along at least portions of the lengths thereof). Insulative housing 585 is formed via overmolding, e.g., with one or multiple-shot overmolds, or is otherwise configured, and serves to capture and retain structural body 582, jaw liner 584, and first and second electrically-conductive plates 586a, 586b in position relative to one another. Insulative housing 585 and/or electrically-conductive plates 586a, 586b are not limited to the configuration illustrated in FIG. 3C but, rather, may define any suitable configuration to achieve a desired height, curvature, protruding distance, angle, etc. of electrically-conductive plates 586a, 586b relative to one another and/or the tissue contacting surface of jaw liner 584, e.g., to achieve any of the configurations detailed herein with respect to other aspects.

Referring back to FIGS. 2A and 2B, jaw liner 184 is shaped complementary to cavity 185c, e.g., defining a T-shaped configuration, for receipt and retention therein and is fabricated from an electrically insulative, compliant material such as, for example, polytetrafluoroethylene (PTFE). The compliance of jaw liner 184 enables blade 162 to vibrate while in contact with jaw liner 184 without damaging components of ultrasonic surgical instrument 100 (FIG. 1A) and without compromising the hold on tissue clamped between jaw member 164 and blade 162. The insulation of jaw liner 184 maintains electrical isolation between blade 162 and structural body 182 of jaw member 164, thereby inhibiting shorting.

Jaw liner 184 includes a tissue contacting surface 188 that is substantially planar (not withstanding gripping teeth and/or indentations formed therein). Tissue contacting surface 188 defines a plane “P2.” Plane “P2” is substantially parallel with a transverse plane “P1.” Plane “P1” is tangential to free ends 186 of uprights 185b of structural body 182. Planes “P1” and “P2” may define a gap distance therebetween, e.g., wherein plane “P2” is recessed within jaw member 164 as compared to plane “P1,” of from about 0.001 inches to about 0.010 inches; in other aspects, from about 0.002 inches to about 0.005 inches; and in still other aspects, of about 0.003 inches. In other configurations, planes “P1” and “P2” are coplanar.

Tissue contacting surface 188 of jaw liner 184 may define a width of from about 0.030 to about 0.70 inches; in other aspects, from about 0.050 to about 0.054 inches; and, in still other aspects, of about 0.052 inches. The width of tissue contacting surface 188 is also substantially the lateral spacing between uprights 185b of structural body 182 (defined between the interior surfaces thereof). Tissue contacting surface 188 may further define a length of about 0.56 inches. Tissue contacting surface 188 may define a surface area of from about 0.020 in² to about 0.040 in²; in other aspects, from about 0.025 in² to about 0.035 in²; and, in still other aspects, about 0.028 in². As pressure is force per unit area, jaw clamping pressure may be stated as jaw clamping force (as detailed above) divided by the surface are of tissue contacting surface 188 (with the assumption that tissue contacts the entire surface area). The jaw clamping pressure applied to tissue may be from about 35 psi to about 285 psi; in other aspects, from about 70 psi to about 180 psi; and in still other aspects from about 90 psi to about 160 psi.

Referring generally to FIGS. 1A, 2A, and 2B, as noted above, end effector assembly 160 is configured for use in an ultrasonic mode of operation and/or one or more electrosurgical modes of operation. The ultrasonic and electrosurgical operating modes may be utilized together, e.g., simultaneously, overlapping, sequentially, etc., and/or may be utilized separately. With respect to the ultrasonic mode of operation, upon activation, an ultrasonic drive signal is provided from surgical generator 200 to ultrasonic transducer 140 to generate ultrasonic energy that is transmitted from ultrasonic transducer 140 along waveguide 154 to blade 162 to thereby vibrate blade 162 at a velocity for treating tissue in contact with or adjacent to blade 162. More specifically, in the ultrasonic mode of operation: ultrasonic energy may be supplied to blade 162 to treat, e.g., seal and/or transect, tissue clamped between tissue contacting surface 171 of blade 162 and tissue contacting surface 188 of jaw liner 184 of jaw member 164; ultrasonic energy may be supplied to blade 162 to treat, e.g., transect, perform an otomy, backscore, etc., tissue in contact with or adjacent to tissue contacting surface 171 of blade 162 (with jaw member 164 disposed in the spaced-apart position) or tissue contacting surface 175 of blade 162 (with jaw member 164 disposed in the spaced-apart or approximated position), statically or dynamically; and/or ultrasonic energy may be supplied to blade 162 to treat, e.g., plunge, spot coagulate, etc., tissue utilizing the distal end of blade 162.

The ultrasonic mode of operation may include one or more energy level settings such as, for example, a first, e.g., LOW, setting and a second, e.g., HIGH, setting. Activation button 120 may include multiple activation switches, multiple activation buttons 120 may be provided, a suitable activation algorithm, etc., may be utilized to enable activation between an OFF condition, a first activated condition corresponding to the first energy level setting, e.g., LOW, and a second activated condition corresponding to the second energy level setting, e.g., HIGH. The first and second energy level settings may correspond to different vibration velocities of blade 162. For example, the first energy level setting may correspond to an unloaded velocity of blade 162 of from 2.4 m/s to about 5.0 m/s, in other aspects, from about 3.0 m/s to about 4.2 m/s; and in still other aspects, of about 3.6 m/s. The second energy level setting may correspond to an unloaded velocity of blade 162 of from 7.0 m/s to about 10.0 m/s; in other aspects, from about 7.5 m/s to about 8.5 m/s; and, in still other aspects, of about 8.0 m/s.

The one or more electrosurgical energy modes of operation may include bipolar electrosurgical modes and/or monopolar electrosurgical modes. In order to enable bipolar electrosurgical modes, structural body 182 and waveguide 154 are adapted to connect to a source of electrosurgical energy, e.g., generator 200. For monopolar electrosurgical modes, structural body 182 and/or waveguide 154 are adapted to connect to generator 200. The bipolar and/or monopolar electrosurgical modes may be tissue treating modes and/or sensing modes, and may be utilized together with one another and/or the ultrasonic modes, e.g., simultaneously, overlapping, sequentially, etc., and/or separately from one and/or the ultrasonic modes.

End effector assembly 160, more specifically, may be configured for use in a bipolar electrosurgical tissue treatment mode of operation, a bipolar electrosurgical sensing mode of operation, a monopolar electrosurgical tissue treatment mode of operation, and/or a monopolar sensing mode of operation. With respect to bipolar electrosurgical tissue treatment, bipolar electrosurgical energy is conducted between first tissue contacting surface 171 of blade 162 and structural body 182 of jaw member 164 to treat, e.g., seal, tissue clamped between first tissue contacting surface 171 and jaw liner 184.

Bipolar electrosurgical tissue treatment may be utilized simultaneously, or otherwise in cooperation with, the ultrasonic mode of operation, e.g., in the first energy level setting, to facilitate treating, e.g., sealing, tissue. Other suitable configurations for bipolar electrosurgical tissue treatment are also contemplated. Bipolar electrosurgical tissue treatment energy, e.g., simultaneously with the ultrasonic mode of operation at the first energy level setting, may be provided at a constant voltage. The constant voltage may be an applied voltage (the voltage applied to tissue; not the voltage output from generator 200) of from about 20 Vrms to about 45 Vrms; in other aspects, from about 25 Vrms to about 40 Vrms; and in still other aspects, from about 30 Vrms to about 35 Vrms. Feedback-based control of output and/or applied electrical properties may be utilized to maintain constant voltage. The constant voltage may be provided at between about 200 kHz and about 400 kHz. The power draw during constant voltage output may be between about 0 W to about 20 W.

With respect to bipolar electrosurgical sensing, an electrical signal is conducted between first tissue contacting surface 171 of blade 162 and structural body 182 of jaw member 164 to enable generator 200 to ascertain one or more properties such as, for example, current, voltage, power, impedance, slopes of these properties, etc. The electrical signal may be the supply of electrosurgical tissue treatment energy or a separate sensing signal and may be utilized before, during, intermittently, and/or after the supply of electrosurgical tissue treatment energy and/or the supply of ultrasonic tissue treatment energy, or separately therefrom. The property(s) sensed during bipolar electrosurgical sensing may be utilized for identifying tissue type, identifying tissue thickness, identifying tissue compressibility, identifying tissue composition (vascular tissue, organ tissue, muscle tissue, etc.), feedback-based control, etc.

Monopolar electrosurgical tissue treatment involves the supply of electrosurgical energy from blade 162 (with jaw member 164 un-energized), from jaw member 164 (with blade 162 un-energized), or from both blade 162 and jaw member 164 (with both energized to the same potential) to tissue to treat, e.g., transect and/or spot coagulate, tissue. Monopolar electrosurgical tissue treatment utilizes a remote return electrode device, e.g., return pad 510 of device 500 (see FIG. 1A) attached to the patient's skin, to safely return energy to generator 200.

Monopolar electrosurgical sensing enables blade 162 (and/or jaw member 164) to function as a monitoring probe, transmitting an electrosurgical signal to tissue such as, for example, for critical anatomical structure identification, nerve monitoring, nearby instrument detection, etc. Monopolar electrosurgical sensing may also be utilized to identify tissue properties, for feedback-based control, etc. in open-jaw conditions, e.g., wherein the monopolar electrosurgical sensing signal is transmitted from blade 162 to tissue and returned via return pad 510 of device 500 (see FIG. 1A). Monopolar electrosurgical sensing may be utilized together with the ultrasonic mode of operation and/or the monopolar electrosurgical tissue treatment mode, e.g., simultaneously, overlapping, sequentially, etc., and/or separately therefrom.

With additional reference to FIG. 4, as noted above, structural body 182 of jaw member 164 and/or blade 162 are adapted to connect to a source of electrosurgical energy for use in bipolar and/or monopolar electrosurgical modes of operation. In order to supply electrosurgical energy to blade 162, one of the electrical lead wires 199 extending from cable assembly 190 into housing 112 is electrically connected to waveguide 154, e.g., via a slip ring connection (not shown) to enable rotation of waveguide 154 relative to housing 112. Thus, electrosurgical energy may be conducted from generator 200, through the electrical lead wire 199, and through waveguide 154 to blade 162. Other configurations are also contemplated.

In order to supply electrosurgical energy to structural body 182 of jaw member 164, one of the electrical lead wires 199 extending from cable assembly 190 into housing 112 is electrically connected to one of the sleeves 152, 153 of elongated assembly 150, e.g., inner support sleeve 153, within housing 112, e.g., via a slip ring connection (not shown), to enable rotation of sleeve 153 relative to housing 112. Inner support sleeve 153, in turn, is electrically coupled to structural body 182 of jaw member 164 via direct contact between pivot bosses 183c of proximal flanges 183a, 183b of structural body 182 and inner support sleeve 153. Thus, electrosurgical energy may be conducted from generator 200, through the electrical lead wire 199, and through inner support sleeve 153 to structural body 182 of jaw member 164. First and second insulators 157, 159 are provided to electrically isolate waveguide 154, inner support sleeve 153, and outer drive sleeve 152 from one another. Insulators 157, 159 may be configured as sheaths, spaced-apart rings, or in any other suitable manner so as to maintain electrical isolation. Waveguide 154, inner support sleeve 153, and outer drive sleeve 152 may be concentrically disposed relative to one another. Other configurations are also contemplated.

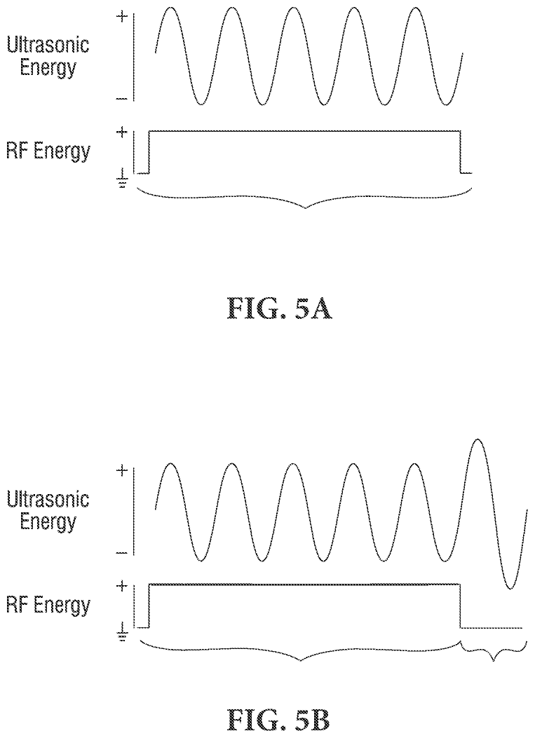

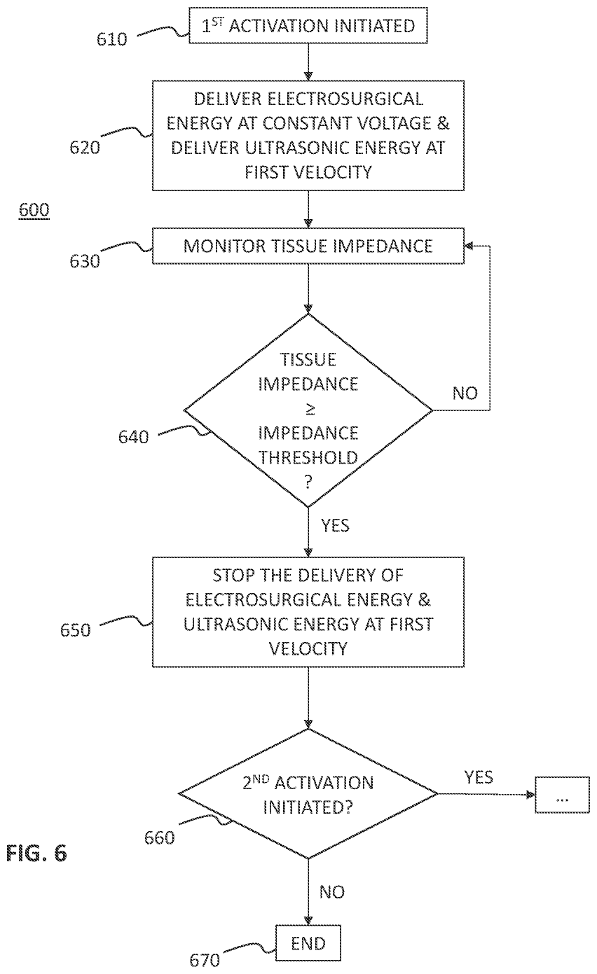

Turning to FIGS. 5A and 6, in conjunction with FIGS. 1A, 2A, and 3, as noted above, the bipolar electrosurgical tissue treatment mode of operation and the ultrasonic mode of operation may be activated simultaneous to seal tissue. Although the methods below are detailed with respect to surgical system 10 (FIG. 1A), it is understood that these methods are equally applicable for use with any of the other surgical systems detailed herein or other suitable surgical system.

In order to seal tissue, tissue is first clamped between blade 162 and jaw member 164 in the approximated position of jaw member 164 such that tissue is held by and in contact with tissue contacting surface 188 of jaw liner 184 and free ends 186 of uprights 185b of structural body 182 on the jaw side of tissue, and first tissue contacting surface 171 of blade 162 on the blade side of tissue.

With respect to simultaneous use of the bipolar electrosurgical tissue treatment mode of operation and the ultrasonic mode of operation to seal tissue, as indicated at 600 (FIG. 6), a first activation is effected at 610 (FIG. 6), e.g., via actuating activation button 120 to a first activated position, with tissue clamped between blade 162 and jaw member 164 as detailed above. Upon the first activation, generator 200 supplies bipolar electrosurgical energy to blade 162 and structural body 182 of jaw member 164 at a constant voltage and ultrasonic energy to blade 162 at a first velocity, as indicated at 620 (FIG. 6). As a result, electrosurgical energy is applied to the clamped tissue via conduction between first tissue contacting surface 171 of blade 162 and free ends 186 of uprights 185b of structural body 182 while ultrasonic energy is applied to the clamped tissue via blade 162.

Tissue impedance is monitored (at 630 (FIG. 6)) during the simultaneous application of electrosurgical and ultrasonic energy and this energy delivery is continued until it is determined that tissue impedance has reaches a threshold impedance. The threshold impedance may be a fixed value, e.g., from about 500 ohms to about 1500 ohms, or may be dynamically determined, e.g., a multiplier (3-10× for example) of the minimum impedance detected throughout the first activation, a multiplier (2-5×, for example) of the average impedance detected over the first activation, or in any other suitable manner. Tissue impedance may be determined by generator 200, e.g., via electrosurgical sensing by sensing the current from generator 200 needed to maintain the constant voltage. More specifically, it is determined at 640 (FIG. 6) whether tissue impedance is equal to or greater than the threshold impedance. If the tissue impedance is not equal to or greater than the threshold impedance (“NO” at 640 (FIG. 6)), the simultaneous application of electrosurgical and ultrasonic energy continues. If, on the other hand, the tissue impedance is equal to or greater than the threshold impedance (“YES” at 640 (FIG. 6)), the supply of electrosurgical and ultrasonic energy is terminated at 650 (FIG. 6) as it is determined that the tissue has been sealed. Alternatively, the method may automatically skip to 710 or 760 once the tissue impedance is equal to or greater than the threshold impedance (“YES” at 640 (FIG. 6)). A notification, e.g., an audible, visual, tactile, and/or other suitable indicator, may be provided to alert a user that the tissue has been sealed, in addition or as an alternative to terminating energy.

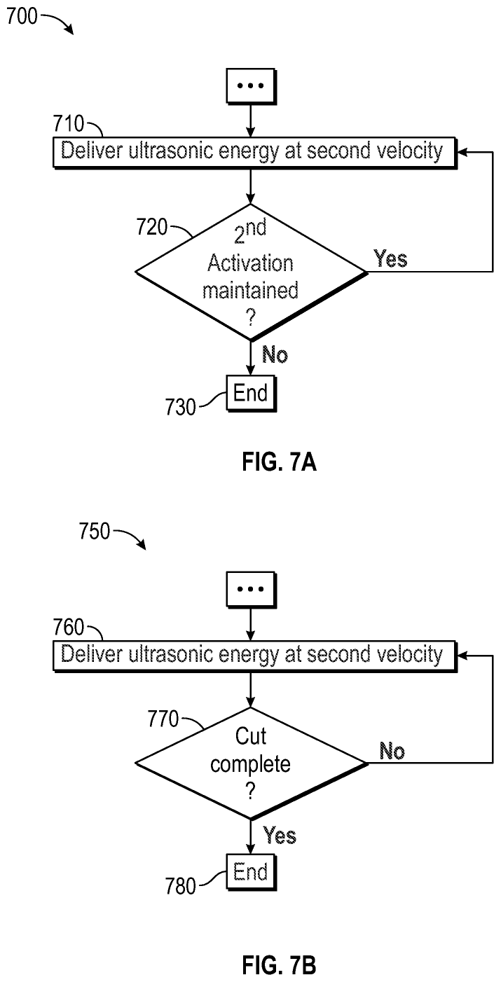

Once tissue has been sealed, it is determined whether the second activation has been effected at 660 (FIG. 6), e.g., whether activation button 120 has been actuated to a second activated position. If the second activation has not been effected, the method ends at 670 (FIG. 6). On the other hand, if the second activation has been effected, the method continues to 700 or 750 (FIGS. 7A and 7B).

Turning to FIGS. 5B and 7A-7B, in conjunction with FIGS. 1A, 2A, and 3, after the simultaneous application of electrosurgical and ultrasonic energy to seal tissue, ultrasonic energy may be utilized to transect the sealed tissue, if so chosen (as indicated by initiating the second activation at 670 (FIG. 6)). Referring to FIG. 7A, once tissue is sealed and the second activation is effected (at 670 (FIG. 6)), generator 200 supplies ultrasonic energy to blade 162 at a second velocity, greater than the first velocity, as indicated at 710 (FIG. 7A); electrosurgical energy is not supplied. Ultrasonic energy is supplied to blade 162 at the second velocity until the second activation is stopped, e.g., until activation button 120 is released, as indicated at 720 (FIG. 7A). That is, if the second activation is maintained (“YES” at 720 (FIG. 7A)), ultrasonic energy is continually supplied to blade 162 at the second velocity. On the other hand, if the second activation is stopped (“NO” at 720 (FIG. 7A)), the supply of ultrasonic energy is terminated at 730 (FIG. 7A).

Referring to FIG. 7B, as an alternative to manually terminating the supply of ultrasonic energy, ultrasonic energy may be terminated based upon a cut complete determination, e.g., a determination that the sealed tissue has been fully transected. More specifically, generator 200 supplies ultrasonic energy to blade 162 at the second velocity, as indicated at 760 (FIG. 7B), until it is determined that the sealed tissue has been fully transected. That is, if it is determined that the transection is not complete (“NO” at 770 (FIG. 7B)), ultrasonic energy is continually supplied to blade 162 at the second velocity. On the other hand, if it is determined that the transection is complete (“YES” at 770 (FIG. 7B)), the supply of ultrasonic energy is terminated at 780 (FIG. 7B). The determination that the sealed tissue has been fully transected may be made by generator 200 based on electrosurgical sensing, e.g., monitoring tissue impedance, and/or ultrasonic feedback, e.g., monitoring load, frequency, drive signal power, current, voltage, etc., or in any other suitable manner. An audible, visual, tactile, and/or other suitable indicator may be provided to alert a user that the tissue has been transected.

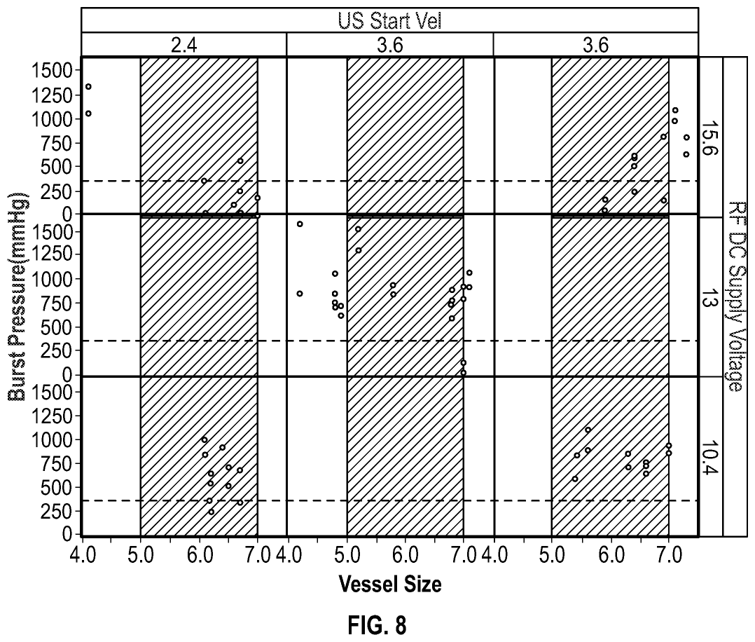

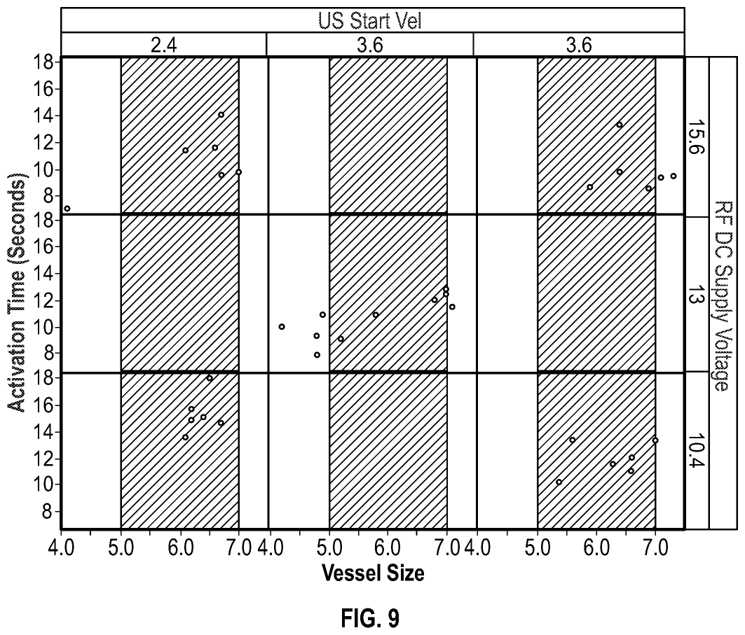

Turning to FIGS. 8 and 9, in conjunction with FIGS. 2A and 2B, in order to effectively and efficiently seal tissue with the simultaneous application of electrosurgical and ultrasonic energy (and to transect sealed tissue using ultrasonic energy), several variables must be taken into consideration such as, for example, the jaw clamping force (or jaw clamping pressure) applied to tissue clamped between jaw member 164 and blade 162, the constant voltage setting, and the blade velocity setting. The configuration and spacing between the free ends 186 of uprights 185b, the offset, e.g., recess, of tissue-contacting surface 188 of jaw liner 184 relative to the free ends 186 of uprights 185b, and the configuration and dimensions of blade 162 also impact tissue sealing. Various exemplary values and/or ranges of these variables are detailed above. Further, the resultant effect on tissue sealing of some or all of these variables is interdependent upon other(s) of these variables, as demonstrated below. As such, the present disclosure specifically includes any and all combinations of these values and/or ranges as well as any and all ratios and/or ratio ranges of the values and/or ranges of two or more of the variables.

FIG. 8 provides a plot of experimental results of burst pressure (in mmHg) of a sealed vessel as a function of the vessel size (diameter, in mm) and the ultrasonic energy setting (velocity, in m/s) and electrosurgical energy setting (RF supply voltage, VDC) used to seal the vessel. More specifically, vessels of various different diameters, e.g., from about 4.0 mm to about 7.0 mm, were sealed using simultaneous application of electrosurgical and ultrasonic energy at various different ultrasonic and electrosurgical energy settings, e.g., blade velocities of 2.4 m/s, 3.0 m/s, and 3.6 m/s, and supply voltages of 10.4 VDC, 13.0 VDC, and 15.6 VDC. The horizontal dashed lines at about 360 mmHg indicate a threshold burst pressure. It is noted that the supply voltage is the voltage output from the generator, not the voltage applied to tissue. As seen from the results presented in FIG. 8, the quality of the resultant tissue seal (as determined by burst pressure) is dependent upon both the ultrasonic energy setting and the electrosurgical energy setting. That is, if either or both of the ultrasonic energy setting or the electrosurgical energy setting are too low or too high, seal quality may suffer. Appropriately selected ultrasonic and electrosurgical energy settings (such as those detailed above), on the other hand, can yield consistent and reliable seals.

FIG. 9 is a plot of experimental results of activation time (in seconds) required to seal a vessel as a function of vessel size (diameter, in mm) and ultrasonic energy setting (velocity, in m/s) and electrosurgical energy setting (RF supply voltage, VDC) used to seal the vessel. More specifically, vessels of various different diameters, e.g., from about 4.0 mm to about 7.0 mm, were sealed using simultaneous application of electrosurgical and ultrasonic energy at various different ultrasonic and electrosurgical energy settings, e.g., blade velocities of 2.4 m/s, 3.0 m/s, and 3.6 m/s, and supply voltages of 10.4 VDC, 13.0 VDC, and 15.6 VDC. It is again noted that the supply voltage is the voltage output from the generator, not the voltage applied to tissue. As seen from the results presented in FIG. 9, the required activation time is dependent upon both the ultrasonic energy setting and the electrosurgical energy setting. Generally, lower ultrasonic and/or electrosurgical energy settings (particularly with respect to the ultrasonic energy setting) require longer activation times. However, the desire to lower activation time must be balanced with the quality of the resultant seal (see FIG. 8). Appropriately selected ultrasonic and electrosurgical energy settings (such as those detailed above) can yield consistent and reliable seals while minimizing activation time.

While several embodiments of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular embodiments. Those skilled in the art will envision other modifications within the scope and spirit of the claims appended hereto.

1. A method of treating tissue, comprising:
clamping tissue between an ultrasonic blade and a jaw member;
simultaneously:
transmitting ultrasonic energy to the ultrasonic blade to vibrate the ultrasonic blade at a first blade velocity, thereby heating the clamped tissue; and
supplying electrosurgical energy, at a constant voltage, to the jaw member and the ultrasonic blade at different potentials such that the electrosurgical energy is conducted therebetween and through the clamped tissue to heat the clamped tissue;
monitoring an impedance of the clamped tissue during the simultaneous transmission of ultrasonic energy and supply of electrosurgical energy; and
at least one of:
terminating the simultaneous transmission of ultrasonic energy and supply of electrosurgical energy when the clamped tissue is sealed, as indicated by the impedance of the clamped tissue being equal to or greater than a threshold impedance; or
outputting a notification when the clamped tissue is sealed, as indicated by the impedance of the clamped tissue being equal to or greater than the threshold impedance.

2. The method according to claim 1, wherein the first blade velocity is from about 2.4 m/s to about 5.0 m/s.

3. The method according to claim 1, wherein the first blade velocity is from about 3.0 m/s to about 4.2 m/s.

4. The method according to claim 1, wherein the first blade velocity is about 3.6 m/s.

5. The method according to claim 1, wherein the constant voltage is an applied voltage of from about 20 Vrms to about 45 Vrms.

6. The method according to claim 1, wherein the constant voltage is an applied voltage of from about 25 Vrms to about 40 Vrms.

7. The method according to claim 1, wherein the constant voltage is an applied voltage of from about 30 Vrms to about 35 Vrms.

8. The method according to claim 1, further comprising, after terminating the simultaneous transmission of ultrasonic energy and supply of electrosurgical energy or outputting the notification:
transmitting ultrasonic energy to the ultrasonic blade to vibrate the ultrasonic blade at a second blade velocity greater than the first blade velocity to transect the sealed tissue.

9. The method according to claim 8, wherein the second blade velocity is from about 7.0 m/s to about 10.0 m/s.

10. The method according to claim 8, wherein the second blade velocity is from about 7.5 m/s to about 8.5 m/s.

11. The method according to claim 8, wherein the second blade velocity is about 8.0 m/s.

12. The method according to claim 8, further comprising terminating the transmission of ultrasonic energy to vibrate the ultrasonic blade at the second blade velocity when it is determined that transection of the sealed tissue is complete.

13. The method according to claim 1, wherein the jaw member includes a body defining first and second radiused surfaces and a jaw liner defining a tissue contacting surface disposed between the first and second radiused surfaces, wherein the tissue contacting surface opposes the ultrasonic blade when clamping tissue therebetween, and wherein supplying the electrosurgical energy includes conducting the electrosurgical energy between the ultrasonic blade and the first and second radiused surfaces.

14. The method according to claim 13, wherein the first and second radiused surfaces define radii of curvature of from about 0.003 inches to about 0.012 inches.

15. The method according to claim 13, wherein the first and second radiused surfaces define radii of curvature of from about 0.005 inches to about 0.010 inches.

16. The method according to claim 13, wherein the first and second radiused surfaces define radii of curvature of about 0.008 inches.

17. The method according to claim 13, wherein a first plane is tangential to the first and second radiused surfaces and the tissue contacting surface defines a second plane, the second plane recessed relative to the first plane a distance of from about 0.001 inches to about 0.010 inches.

18. The method according to claim 13, wherein a first plane is tangential to the first and second radiused surfaces and the tissue contacting surface defines a second plane, the second plane recessed relative to the first plane a distance of from about 0.002 inches to about 0.005 inches.

19. The method according to claim 13, wherein a first plane is tangential to the first and second radiused surfaces and the tissue contacting surface defines a second plane, the second plane recessed relative to the first plane a distance of about 0.003 inches.

20. The method according to claim 1, wherein the ultrasonic blade defines a tissue contacting surface having first and second angled or arcuate surface portions meeting at an apex configured to oppose the jaw member when clamping tissue therebetween.

21-28. (canceled)
